(19) United States
(12) Patent Application Publication
(10) Pub. No.: US 2014/0228265 A1
(43) Pub. Date: Aug. 14, 2014

(54) Bridged Alkylphenol Compounds

(71) Applicant: The Lubrizol Corporation, Wickliffe, OH (US)

(72) Inventors: Stephen J. Cook, Belper (GB); Christopher L. Friend, Willoughby, OH (US); Gary M. Walker, Allestree (GB)

(73) Assignee: The Lubrizol Corporation, Wickliffe, OH (US)

(21) Appl. No.: 14/346,335

(22) PCT Filed: Oct. 16, 2012
(86) PCT No.: PCT/US2012/060389

§ 371 Date: Mar. 21, 2014

Related U.S. Application Data
(60) Provisional application No. 61/549,286, filed on Oct. 20, 2011.

(51) Int. Cl. C10M 141/10; C10M 135/30; C10M 141/08; C07C 323/20

(52) U.S. Cl. CPC C10M 141/10; C07C 323/20; C10M 135/30; C10M 141/08
USPC 508/378; 568/48; 508/572; 508/390

(57) ABSTRACT

A bridged dimeric or oligomeric phenolic compound comprising: at least one monomer unit (a) of phenol or an alkyl-substituted phenol wherein the alkyl group contains 1 to 8 carbon atoms, or mixtures thereof; at least one monomer unit (b) of an aliphatic hydrocar-byl-substituted phenol wherein the aliphatic hydrocarbyl group contains at least about 25 carbon atoms, or mixtures thereof; and at least one sulfur-containing or carbon-containing bridging group; or a salt of said oligomeric material; wherein the average number of carbon atoms in said alkyl groups and said aliphatic hydrocarbyl groups is 10 to 100.

### BACKGROUND OF THE INVENTION

The disclosed technology relates to bridged hydrocarbyl- (e.g., alkyl-) phenol compounds and their salts, free from or substantially free from C-12 alkyl phenol moieties. Such compounds and their salts are useful as lubricant additives.

Phenol-based detergents are known. Among these are phenates based on phenolic monomers, linked with sulfur bridges or alkylene bridges such as methylene linkages derived from formaldehyde. The phenolic monomers themselves are typically substituted with an aliphatic hydrocarbyl group to provide a measure of oil solubility. The hydrocarbyl groups may be alkyl groups, and, historically, dodecylphenol (or propylene tetramer-substituted phenol) has been widely used. An early reference to basic sulfurized polyvalent metal phenates is U.S. Pat. No. 2,680,96, Walker et al., Jun. 1, 1954; see also U.S. Pat. No. 3,372,116, Meinhardt, Mar. 6, 1968.

Recently, however, certain alkylphenols and products prepared from them have come under increased scrutiny due to their association as potential endocrine disruptive materials. In particular, alkylphenol detergents which are based on oligomers of C₁₂ alkyl phenols may contain residual monomeric C₁₂ alkyl phenol species. There is interest, therefore, in developing alkyl-substituted phenate detergents, for uses in lubricants, fuels, and as industrial additives, which contain a reduced or eliminated amount of dodecylphenol component and other substituted phenols having alkyl substituents of 10 to 15 carbon atoms. Nevertheless, it is desirable that the products should have similar oil-solubility parameters as phenates prepared from C₁₀₋₁₅ alkylphenols. Preparing phenate detergents from unsubstituted phenol alone or from cresols alone (methyl phenols) is undesirable because such materials will typically have only very limited oil-solubility. On the other hand, preparing phenate detergents from polyisobutene-substituted phenol alone is undesirable because such materials have a tendency to impart excessive viscosity to lubricants and may also be difficult to handle due to their high viscosity.

There have been several efforts to prepare phenate detergents that do not contain C₁₂ alkyl phenols. U.S. Pat. No. 7,435,709, Stonebraker et al., Oct. 14, 2008, discloses a linear alkylphenol derived detergent substantially free of endocrine disruptive chemicals. It comprises a salt of a reaction product of (1) an olefin having at least 10 carbon atoms, where greater than 90 mole % of the olefin is a linear C₂₀-C₃₀ n-alpha olefin, and wherein less than 10 mole % of the olefin is a linear olefin of less than 20 carbon atoms, and less than 5 mole % of the olefin a branched chain olefin of 18 carbons or less, and (2) a hydroxyaromatic compound.

U.S. Application 2011/0190185, Sinquin et al., Aug. 4, 2011, discloses an overbased salt of an oligomerized alkylhydroxyaromatic compound. The alkyl group is derived form an olefin mixture comprising propylene oligomers having an initial boiling point of at least about 195° C. and a final boiling point of greater than 325° C. The propylene oligomers may contain a distribution of carbon atoms that comprise at least about 50 weight percent of C₁₄ to C₂₀ carbon atoms.

U.S. Application 2011/0124539, Sinquin et al., May 26, 2011, discloses an overbased, sulfurized salt of an alkylated hydroxyaromatic compound. The alkyl substituent is a residue of at least one isomerized olefin having from 15 to about 99 wt. % branching. The hydroxyaromatic compound may be phenol, cresols, xylenols, or mixtures thereof.

U.S. Application 2011/0118160, Campbell et al., May 19, 2011, discloses an alkylated hydroxyaromatic compound substantially free of endocrine disruptive chemicals. An alkylated hydroxyaromatic compound is prepared by reacting a hydroxyaromatic compound with at least one branched olefinic propylene oligomer having from about 20 to about 80 carbon atoms. Suitable hydroxyaromatic compounds include phenol, catechol, resorcinol, hydroquinone, pyrogallol, cresol, and the like.

U.S. Application 2010/0029529, Campbell et al., Feb. 4, 2010, discloses an overbased salt of an oligomerized alkylhydroxyaromatic compound. The alkyl group is derived from an olefin mixture comprising propylene oligomers having an initial boiling point of at least about 195° C. and a final boiling point of no more than about 325° C. Suitable hydroxyaromatic compounds include phenol, catechol, resorcinol, hydroquinone, pyrogallol, cresol, and the like.

U.S. Application 2008/0269351, Campbell et al., Oct. 30, 2008, discloses an alkylated hydroxyaromatic compound substantially free of endocrine disruptive chemicals, prepared by reacting a hydroxyaromatic compound with a branched olefinic oligomer having from about 20 to about 80 carbon atoms.

Other general technology includes that of U.S. Pat. No. 6,310,009, Carrick et al., Oct. 30, 2001, which discloses salts of the general structure

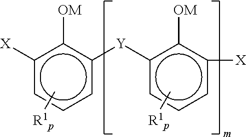

where R¹ may be an alkyl group of 1 to 60 carbon atoms, e.g., 9 to 18 carbon atoms. It is understood that R¹ will normally comprise a mixture of various chain lengths, so that the foregoing numbers will normally represent an average number of carbon atoms in the R¹ groups (number average).

The disclosed technology, therefore, solves the problem of providing a phenolic material with appropriate oil solubility, viscosity performance, and detergency (characteristic of moderate chain length alkyl groups) but free from or substantially free from C₁₂ alkyl phenol moieties.

### SUMMARY OF THE INVENTION

The disclosed technology provides a bridged phenolic compound comprising an oligomeric material comprising at least one monomer unit of (a) phenol or an alkyl-substituted phenol wherein the alkyl group contains 1 to 8 carbon atoms, or mixtures thereof; and at least one monomer unit of (b) an aliphatic hydrocarbyl-substituted phenol wherein the aliphatic hydrocarbyl group contains at least 25 carbon atoms, or mixtures thereof; or a salt of said oligomeric material; wherein the average number of carbon atoms in said alkyl groups and said aliphatic hydrocarbyl groups is 10 to 100 (or 12 to 50, or 14 to 36 or 14 to 20 or 18 to 36); and wherein said oligomeric material is substantially free of (or entirely free of, or contains less than 5 percent or 3 percent or 1 percent or 0.3 percent or 0.1 percent by mole of) monomer units of C₁₂-alkyl phenol. By “monomer units of C₁₂ alkyl phenol” is meant to include both the salts and the hydroxy forms as contained within the oligomeric material. In certain embodiments the materials presently disclosed will also be substantially free of (or entirely free of, or contain less than 0.05 percent or 0.01 percent or 0.001 percent by weight) of C₁₂ alkyl phenol in the free monomeric form.

In another expression, the disclosed technology provides a bridged dimeric or oligomeric phenolic compound comprising an oligomeric material comprising: at least one monomer unit (a) of phenol or an alkyl-substituted phenol wherein the alkyl group contains 1 to 8 carbon atoms, or mixtures thereof; and at least one monomer unit (b) of an aliphatic hydrocarbyl-substituted phenol, wherein the aliphatic hydrocarbyl group contains at least 25 carbon atoms, or mixtures thereof; and at least one sulfur-containing or carbon-containing bridging group; or a salt of said oligomeric material; wherein the average number of carbon atoms in said alkyl groups and said aliphatic hydrocarbyl groups is 10 to 100 (or 12 to 50, or 14 to 36 or 14 to 20 or 18 to 36).

The present technology also provides a product prepared by reacting at least one monomer (a) of phenol or an alkyl-substituted phenol wherein the alkyl group contains 1 to 8 carbon atoms, or mixtures thereof; and at least one monomer (b) of an aliphatic hydrocarbyl-substituted phenol wherein the aliphatic hydrocarbyl group contains at least about 25 carbon atoms, or mixtures thereof; with a bridging agent comprising sulfur or an aldehyde or ketone, wherein the amounts of (a) and (b) are such that the average number of carbon atoms in said alkyl groups and said aliphatic hydrocarbyl groups is about 10 to about 100 (or about 12 to about 50, or about 14 to about 36 or about 14 to about 20 or about 18 to about 36).

The disclosed technology also provides a lubricant comprising an oil of lubricating viscosity and said bridged phenolic compound, as well as a method of lubricating a mechanical device with said lubricant.

### DETAILED DESCRIPTION OF THE INVENTION

Various preferred features and embodiments will be described below by way of non-limiting illustration.

One of the materials of the presently disclosed technology is a bridged phenolic compound. Such materials in general, their methods of preparation, and use in lubricants are well known from, for instance, the above-referenced U.S. Pat. No. 2,680,096, Walker et al. They may be prepared starting from phenol or, alternatively, a short chain alkyl phenol such as cresol (o-, m-, or p-methylphenol), or mixtures thereof, any of which are readily available as starting materials. The alkylation of phenol and its homologues is well known, typically by catalyzed reaction of an olefin, often an α-olefin, with phenol (or with cresol or another homologue, as the case may be). Alkylation of phenol is described in greater detail in the Kirk-Othmer Encyclopedia of Chemical Technology, third edition (1978) vol. 2, pages 82-86, John Wiley and Sons, New York.

Linking of alkyl (or more generally, hydrocarbyl) phenols to form oligomeric species, is also well known. They may be condensed, for instance, with formaldehyde or with other aldehydes or ketones such as acetone to form methylene (or alkylene) bridged structures, as described on pages 76-77 of the above cited Kirk-Othmer reference. If condensation with an aldehyde or ketone is intended, it is desirable that the aldehyde or ketone not be a C12 species, to avoid the formation of any C12 substituted phenolic materials. In certain embodiments the material is an aldehyde of 8 or fewer carbon atoms, such as 1 to 4, or 1 or 2, or a single carbon atom (formaldehyde). The length of the resulting oligomeric chain of phenolic and alkylene units will depend to some extent on the molar ratio of the reactants, as is well known. Thus an equimolar amount of phenol and formaldehyde will give a condensate with a relatively longer oligomeric chain than that obtained when there is a stoichiometric excess of one species or the other. Under certain conditions, carbon- and oxygen-containing linkages may also be formed, such as those of the general structure —CH₂—O—CH₂— or homologues in which the hydrogens are replaced by alkyl groups. These may be formed by the condensation of more than a single aldehyde or ketone group. Such structures are known, for example, from U.S. Pat. No. 6,310,009, see col 2 lines 14-17 and col. 6 lines 1-45. Thus the linking groups prepared form aldehydes or ketones may be generally described as “carbon-containing” bridging groups, e.g., an alkylene bridge or an ether bridge.

Substituted phenols may also be linked together to make sulfur bridged species, which may include bridges of single sulfur atoms (—S—) or multiple sulfur atoms (e.g., —Sₓ— where x may be 2 to 8, typically 2 or 3). Sulfurized phenols may be prepared by reaction with active sulfur species such as sulfur monochloride or sulfur dichloride as described on pages 79-80 of the Kirk-Othmer reference or with elemental sulfur, as described, for instance, in U.S. Pat. No. 2,680,096. Sulfurization (with sulfur) may be conducted in the presence of a basic metal compound such as calcium hydroxide or calcium oxide, thus preparing a metal salt, as described in greater detail, below. Basic sulfurized phenates and a method for their preparation are also disclosed in U.S. Pat. No. 3,410,798, Cohen, Nov. 12, 1968. The examples and claim 1 thereof disclose a method, comprising reacting at a temperature above about 150° C., (A) a phenol, (B) sulfur, and (C) an alkaline earth base, in the presence of a promoter comprising (D) about 5-20 mole percent, based on the amount of component A, of a carboxylic acid or alkali metal, alkaline earth metal, zinc, or lead salt thereof and (E) as a solvent, a compound of the formula R(OR′)ₓOH, e.g., a polyalkylene glycol. The phenol (A), in turn, may be a hydrocarbyl-substituted phenol which may be prepared by mixing a hydrocarbon and a phenol at a temperature of about 50-200° C. in the presence of a suitable catalyst such as aluminum trichloride (col. 2 line 51 of U.S. Pat. No. 3,410,798, and following text.)

In the present technology, the selection of the alkyl groups is to be made such that there are at least two different types of phenols that are present. One type is a phenol that is unsubstituted by a hydrocarbyl or alkyl group or alternatively contains alkyl groups of only 1 to 8 carbon atoms, typically a single methyl substituent. (Any alkylene bridges are not generally counted as representing an alkyl substituent, in this context.) Methyl phenol, that is, cresol, is available in three isomers, ortho, meta, and para, and is commercially available in any of these isomer or as mixtures thereof. Alkyl phenols having 2 to 8 carbon atoms in the alkyl group(s) are also commercially available and may be prepared, for instance, by heating of phenol with the corresponding olefin or alcohol in the presence of acid. In one embodiment the phenol is unsubstituted phenol. In another embodiment the phenolic compound is cresol. In other embodiments the phenolic compound may have one or more alkyl substituents having 1 to 8 or 1 to 6 or 1 to 4 or 2 to 6 or 2 to 4 or 1 to 2 carbon atoms.

The second phenol component is a phenol substituted by an aliphatic hydrocarbyl group (which may be an alkyl group) of at least 25 carbon atoms, or mixtures of such groups. In certain embodiments such relatively long-chain aliphatic hydrocarbon groups may contain 25 to 200, or 30 to 200, or 35 to 100, or 35 to 80, or 40 to 70 carbon atoms, e.g., at least 25 or at least 30, 32, or 36 carbon atoms. Such substituted phenols are known and may be prepared by alkylation of phenol with a suitable alkylating group such as a polyolefin containing a point of unsaturation. Suitable polyolefins include oligomers or polymers of propylene or of isobutylene. A propylene polymer or oligomer containing 48-54 carbon atoms would contain 16 to 18 propylene monomer units. An isobutylene polymer or oligomer containing 48 to 52 carbon atoms would contain 12 or 13 isobutylene monomer units. Further details of alkylation are disclosed in the above-cited Kirk Othmer reference. The second phenol component may be further unsubstituted (that is, no substituents on the ring other than the phenolic —OH and the relatively long chain aliphatic hydrocarbyl group). Alternatively, the aromatic ring of this component may optionally be further substituted by one or more short chain alkyl groups as described above. For example, the second phenol component may be cresol substituted with the aliphatic hydrocarbyl group of at least 25 carbon atoms, or, alternatively, it may be phenol itself, substituted with the aliphatic hydrocarbyl group of at least 25 carbon atoms.

If the second phenol component is a polyisobutylene-substituted phenol, it may optionally be prepared from a high-vinylidene content polyisobutylene, that is, containing greater than 70 percent or greater than 75 percent terminal vinylidene groups. In other embodiments, the polyisobutylene may contain greater than 70 percent or greater than 75 percent terminal vinylidene groups+terminal α,α-dimethylvinyl groups (containing a 13 double bond).

The average number of carbon atoms in the alkyl and hydrocarbyl substituents of the bridged phenolic compound (or mixture of such individual compounds) will be 10 to 100 or 12 to 50, or 14 to 36 or 14 to 20 or 18 to 36. This represents an overall average of both the longer and shorter types of substituents. This average number may readily be determined by the person of ordinary skill by a consideration of the lengths of the substituents on the constituent substituent phenol components. The presence of phenol units without alkyl or hydrocarbyl substituents will typically be included in the average as contributing zero carbon atoms to the total. For instance, a bridged phenolic compound may be prepared by reacting 1 mole of a C35 (number average) alkyl substituted phenol with two moles of cresol and a bridging agent such as sulfur. The resulting product will have on average about 12 (or 12.33) carbon atoms per alkyl or aliphatic hydrocarbyl group and yet will be free, or substantially free, of dodecylphenol component. A material with similar average molecular weight in the substituents may be prepared by reacting 3 moles of cresol with 1 mole of a C45 average alkyl substituted phenol.

In certain embodiments, the bridged dimeric or oligomeric phenolic compound does contain, or alternatively does not contain unsubstituted phenol units, that is, rings in which the “R” group in the structures (I) through (V), below, is hydrogen. The compound may be completely free or may be substantially free of such units, i.e., containing in the overall composition (which will typically refer to the a mixture of molecules) less than 5 mole percent or less than 2 or 1 or 0.5 or 0.1 mole percent, such as 0.01 to 0.1 mole percent, of such unsubstituted phenol units. If the compound does contain unsubstituted phenol units, then in one embodiment the average number of carbon atoms in the alkyl and hydrocarbyl substituents, as calculated above, will include phenol units as contributing zero carbon atoms to the total average carbon number. For instance, a mixture of 40 mol % of C36-substituted phenol, 53 mol % p-cresol (C1), and 7 mol % phenol may have an average carbon chain number of about 15.

The mole ratio of long-chain hydrocarbyl-substituted phenol (“long”) to short chain alkyl-substituted, or unsubstituted, phenol (“short”) may be the ratio necessary to obtain the average number of carbon atoms as described in the previous paragraph. In certain embodiments, the mole ratio of long:short may be about 1:1, e.g., 0.25:1 to 2:1, or 0.3:1 to 1.5:1 or 0.5:1 to 1.3:1 or 0.8:1 to 1.1:1.

The bridged phenolic compound and the oligomeric material may be represented by the structure

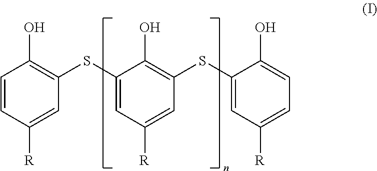

or more generally

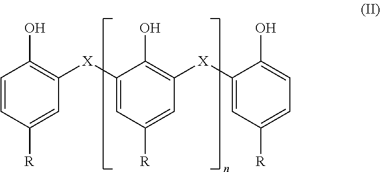

or isomers thereof, wherein each R is independently hydrogen or an aliphatic hydrocarbyl group, provided that at least one R represents an aliphatic hydrocarbyl group containing at least 25 carbon atoms and at least one R represents hydrogen or a hydrocarbyl group of 1 to 8 or 1 to 4 carbon atoms. The average number of carbon atoms in all the R groups, combined, may be 10 to 100 (or 12 to 50, or 14 to 36 or 14 to 20 or 18 to 36). Where the bridging group is listed as “X”, each X may independently a carbon-containing bridge, or an alkylene group, or a methylene group, or a bridge of 1 or more sulfur atoms represented by S_{X}, where x is 1 to 4, especially 1 or 2. In these structures, n may, in certain embodiments, be 0 to 8, or 1 to 6, or 1 to 4, or 2 to 4. That is, the oligomeric material may, in these embodiments, contain 2 to 10 bridged phenolic groups, or 3 to 7, or 3 to 5, or 4 such groups. Since n may be zero, it is evident that throughout this specification, the expression “oligomeric” may be interpreted to include dimeric species. Accordingly, sometimes the expression “dimeric or oligomeric” may be used to express this concept, which may include, as above, as an example, 0 to 8 interior units bracketed by [ ]ₙ or 2 to 10 units overall. In certain embodiments, in the above structure, one or two of the R groups are aliphatic hydrocarbyl groups containing 30 to 200 or 35 to 80 carbon atoms and the remainder of the R groups are methyl groups.

Alternatively, certain of the above embodiments may include

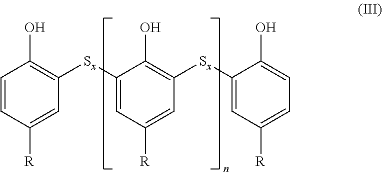

where the groups are as defined above.

A more general representation of the “isomers thereof” as used herein could be written as follows:

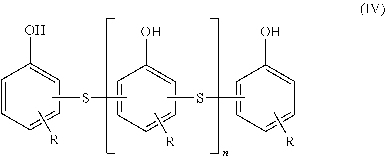

It is to be understood that in alternative embodiments the “S” bridges may represent not only single sulfur atoms but also chains of two or more sulfur atoms, such as one or more disulfide linkages as has been previously discussed. Also, in some embodiments the S bridges may be replaced by alkylene or other carbon-containing bridges.

It will also be recognized by those skilled in the art that the bridging functionality, whether S-, aldehyde-, or ketone-derived, may form additional groups on the terminal phenolic rings, in addition to the internal linkages shown in the above structures, to form one or more “Y” groups as shown in the following illustrative structure:

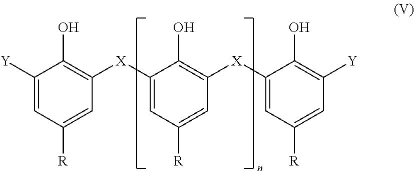

and isomers thereof, where X, R, and n are defined as above and each Y is independently hydrogen or a terminal group derived from sulfur or an aldehyde or ketone. Y groups derived from formaldehyde, for instance, may include —CHO or —CH₂OH groups, as described in greater detail in U.S. Pat. No. 6,310,009, col. 2 lines 14-17 and col. 6 lines 1-45. A Y group derived from sulfur may include —SH or —SSH. Such structures may be considered to be included within the materials “represented by” structures (I) through (IV) above; that is, each structure (I) through (IV) may also implicitly contain one or more terminal Y groups.

The bridged phenolic compound may be present in the form of a salt, in which one or more of the phenolic OH groups is in the anionic form. The metal compounds useful in making the salts are generally any Group 1 or Group 2 metal compounds (CAS version of the Periodic Table of the Elements). Examples include alkali metals such as sodium, potassium, lithium, copper, magnesium, calcium, barium, zinc, and cadmium. In one embodiment the metals are sodium, magnesium, or calcium. Alternatively, the salt may be an ammonium salt or an amine salt, including a quaternary amine salt.

If in the form of a salt, the phenolic functionality may be partially neutralized by the basic material, completely neutralized, or “overbased.” Overbased or superbased salts are generally homogeneous Newtonian systems having by a metal (or other cation) content in excess of that which would be present for neutralization according to the stoichiometry of the metal and the detergent anion. Overbased materials are typically prepared by reacting an acidic material (typically an inorganic acid or lower carboxylic acid, typically carbon dioxide) with a mixture of an acidic organic compound (in this case, the bridged phenolic compound), a reaction medium comprising at least one inert, organic solvent (e.g., mineral oil, naphtha, toluene, xylene) for said acidic organic material, a stoichiometric excess of a metal base, and a promoter such as a phenol or alcohol and optionally ammonia. The acidic organic material will normally have a sufficient number of carbon atoms, for instance, as a hydrocarbyl substituent, to provide a reasonable degree of solubility in oil. The amount of excess metal is commonly expressed in terms of metal ratio, that is, the ratio of the total equivalents of the metal to the equivalents of the acidic organic compound. Further details in the preparation of overbased phenates may be found in U.S. Pat. No. 3,372,116, Meinhardt (see, for instance, Example 1).

Overbased materials (also referred to as overbased detergents) may be characterized by Total Base Number (TBN), the amount of strong acid needed to neutralize all of the material's basicity, expressed as mg KOH per gram of sample. Since overbased detergents are commonly provided in a form which contains diluent oil, for the purpose of this document, TBN is to be recalculated to an oil-free basis. Some useful detergents may have a TBN of 50 to 800, or 80 to 300, or 100 to 280, or 110 to 250, or 120 to 160.

The metal compounds useful in making the basic metal salts are generally those mentioned above in the context of preparing the neutral salts. The anionic portion of the metal compound used to prepare the overbased salt can be, for example, hydroxide, oxide, carbonate, borate, or nitrate.

The amount of the bridged phenolic compound, when it is present as an overbased detergent, may vary depending on the end-use application. When used in a passenger car lubricant it may be present as low as 0.1 weight percent, and when used in a marine diesel cylinder lubricant it may be present in amounts as high as 25 percent by weight of the lubricant. Therefore, suitable ranges may include 0.1 to 25%, or 0.5 to 20%, or 1 to 18% or 3 to 13% or 5 to 10%, or 0.7 to 5 weight percent or 1 to 3 weight percent, all on an oil-free basis Similar overall amounts may also be used if the bridged phenolic compound is not overbased.

Either a single detergent or multiple detergents can be present. If there are multiple detergents, the additional detergents may be additional phenate detergents, or they may be detergents of other types. An example of another types of detergent is a sulfonate detergent, prepared from a sulfonic acid. Suitable sulfonic acids include sulfonic and thiosulfonic acids, including mono or polynuclear aromatic or cycloaliphatic compounds. Certain oil-soluble sulfonates can be represented by R²T(SO3-)a or R³(SO3-)b, where a and b are each at least one; T is a cyclic nucleus such as benzene or toluene; R² is an aliphatic group such as alkyl, alkenyl, alkoxy, or alkoxyalkyl; (R²)-T typically contains a total of at least 15 carbon atoms; and R³ is an aliphatic hydrocarbyl group typically containing at least 15 carbon atoms. The groups T, R², and R³ can also contain other inorganic or organic substituents. In one embodiment the sulfonate detergent may be a predominantly linear alkylbenzenesulfonate detergent having a metal ratio of at least 8 as described in paragraphs [0026] to [0037] of US Patent Application 2005065045. In some embodiments the linear alkyl group may be attached to the benzene ring anywhere along the linear chain of the alkyl group, but often in the 2, 3 or 4 position of the linear chain, and in some instances predominantly in the 2 position.

Another overbased material is an overbased saligenin detergent, other than that which may appear as one of the embodiments of the present invention. Overbased saligenin detergents are commonly overbased magnesium salts which are based on saligenin derivatives. A general example of such a saligenin derivative can be represented by the formula

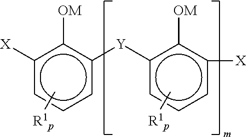

where X is —CHO or —CH₂OH, Y is —CH₂— or —CH₂OCH₂—, and the —CHO groups typically comprise at least 10 mole percent of the X and Y groups; M is hydrogen, ammonium, or a valence of a metal ion (that is, if M is multivalent, one of the valences is satisfied by the illustrated structure and other valences are satisfied by other species such as anions or by another instance of the same structure), R¹ is a hydrocarbyl group of 1 to 60 carbon atoms, m is 0 to typically 10, and each p is independently 0, 1, 2, or 3, provided that at least one aromatic ring contains an R¹ substituent and that the total number of carbon atoms in all R¹ groups is at least 7. When m is 1 or greater, one of the X groups can be hydrogen. In one embodiment, M is a valence of a Mg ion or a mixture of Mg and hydrogen. Saligenin detergents are disclosed in greater detail in U.S. Pat. No. 6,310,009, with special reference to their methods of synthesis (Column 8 and Example 1) and preferred amounts of the various species of X and Y (Column 6). Saligenin detergents may be seen as a species of phenate detergents, and therefore it may be desirable that they be prepared with the selection of R¹ groups made so as to satisfy the requirements in terms of number of carbon atoms as in the bridged phenolic compounds described in greater detail above. (That is, there may be in one embodiment a mixture of long chain and short chain groups in the ranges of 1 to 8 and at least 25 carbon atoms, such that the average number of carbon atoms in the groups is 10 to 100, or other ranges as set forth above and the detergent or the unneutralized compound is substantially free of monomer units of C₁₂ alkyl phenol.)

Salixarate detergents are overbased materials that can be represented by a compound comprising at least one unit of formula (I) or formula (II):

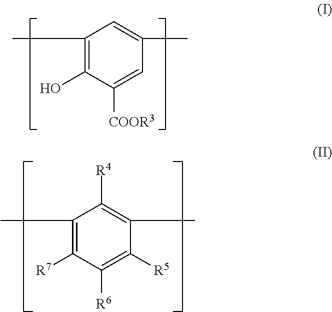

each end of the compound having a terminal group of formula (III) or (IV):

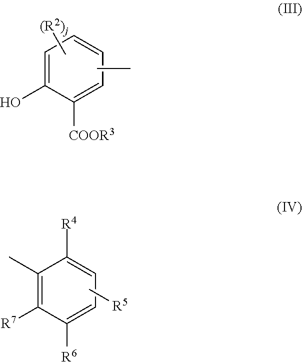

such groups being linked by divalent bridging groups A, which may be the same or different. In formulas (I)-(IV) R³ is hydrogen, a hydrocarbyl group, or a valence of a metal ion; R² is hydroxyl or a hydrocarbyl group, and j is 0, 1, or 2; R⁶ is hydrogen, a hydrocarbyl group, or a hetero-substituted hydrocarbyl group; either R⁴ is hydroxyl and R⁵ and R⁷ are independently either hydrogen, a hydrocarbyl group, or hetero-substituted hydrocarbyl group, or else R⁵ and R⁷ are both hydroxyl and R⁴ is hydrogen, a hydrocarbyl group, or a hetero-substituted hydrocarbyl group; provided that at least one of R⁴, R⁵, R⁶ and R⁷ is hydrocarbyl containing at least 8 carbon atoms; and wherein the molecules on average contain at least one of unit (I) or (III) and at least one of unit (II) or (IV) and the ratio of the total number of units (I) and (III) to the total number of units of (II) and (IV) in the composition is 0.1:1 to 2:1. The divalent bridging group “A,” which may be the same or different in each occurrence, includes —CH₂— and —CH₂OCH₂—, either of which may be derived from formaldehyde or a formaldehyde equivalent (e.g., paraform, formalin). If desired, the salixarate materials may be prepared with a selection of groups R⁵, R⁶, and R⁷ made so as to satisfy the requirements in terms of number of carbon atoms is in the bridged phenolic compounds described in greater detail above.

Salixarate derivatives and methods of their preparation are described in greater detail in U.S. Pat. No. 6,200,936 and PCT Publication WO 01/56968. It is believed that the salixarate derivatives have a predominantly linear, rather than macrocyclic, structure, although both structures are intended to be encompassed by the term “salixarate.”

Glyoxylate detergents are similar overbased materials which are based on an anionic group which, in one embodiment, may have the structure

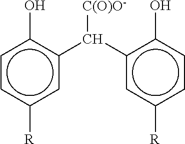

wherein each R is independently an alkyl group containing at least 4 or 8 carbon atoms, provided that the total number of carbon atoms in all such R groups is at least 12 or 16 or 24. Alternatively, each R can be an olefin polymer substituent. The acidic material upon from which the overbased glyoxylate detergent is prepared is the condensation product of a hydroxyaromatic material such as a hydrocarbyl-substituted phenol with a carboxylic reactant such as glyoxylic acid or another omega-oxoalkanoic acid. If desired, the glyoxylate materials may be prepared with a selection of R groups made so as to satisfy the requirements in terms of number of carbon atoms is in the bridged phenolic compounds described in greater detail above. Overbased glyoxylic detergents and their methods of preparation are disclosed in greater detail in U.S. Pat. No. 6,310,011 and references cited therein.

The overbased detergent can also be an overbased salicylate, e,g., an alkali metal or alkaline earth metal salt of a substituted salicylic acid. The salicylic acids may be hydrocarbyl-substituted wherein each substituent contains an average of at least 8 carbon atoms per substituent and 1 to 3 substituents per molecule. The substituents can be polyalkene substituents. In one embodiment, the hydrocarbyl substituent group contains 7 to 300 carbon atoms and can be an alkyl group having a molecular weight of 150 to 2000. Overbased salicylate detergents and their methods of preparation are disclosed in U.S. Pat. Nos. 4,719,023 and 3,372,116.

Other overbased detergents can include overbased detergents having a Mannich base structure, as disclosed in U.S. Pat. No. 6,569,818.

The amount of any supplemental overbased detergent or detergents, if present in a lubricant, may be 0.1 to 20, or 0.5 to 18, or 1, 2, or 3 to 13 percent by weight.

The materials of the disclosed technology are typically employed in an oil to form a composition that may be used as a lubricant. The oil is typically referred to as an oil of lubricating viscosity, also referred to as a base oil. The base oil may be selected from any of the base oils in Groups I-V of the American Petroleum Institute (API) Base Oil Interchangeability Guidelines, namely

| Base | | | | |
|---|---|---|---|---|
| Oil Category | Sulfur (%) | | Saturates (%) | Viscosity Index |
| Group I | >0.03 | and/or | <90 | 80 to 120 |
| Group II | ≦0.03 | and | ≧90 | 80 to 120 |
| Group III | ≦0.03 | and | ≧90 | >120 |

| | |
|---|---|
| Group IV | All polyalphaolefins (PAOs) |
| Group V | All others not included in Groups I, II, III or IV |

Groups I, II and III are mineral oil base stocks. The oil of lubricating viscosity can include natural or synthetic oils and mixtures thereof. Mixture of mineral oil and synthetic oils, e.g., polyalphaolefin oils and/or polyester oils, may be used.

Natural oils include animal oils and vegetable oils (e.g. vegetable acid esters) as well as mineral lubricating oils such as liquid petroleum oils and solvent-treated or acid treated mineral lubricating oils of the paraffinic, naphthenic or mixed paraffinic-naphthenic types. Hydro treated or hydrocracked oils are also useful oils of lubricating viscosity. Oils of lubricating viscosity derived from coal or shale are also useful.

Synthetic oils include hydrocarbon oils and halosubstituted hydrocarbon oils such as polymerized and interpolymerized olefins and mixtures thereof, alkylbenzenes, polyphenyl, alkylated diphenyl ethers, and alkylated diphenyl sulfides and their derivatives, analogs and homologues thereof. Alkylene oxide polymers and interpolymers and derivatives thereof, and those where terminal hydroxyl groups have been modified by, e.g., esterification or etherification, are other classes of synthetic lubricating oils. Other suitable synthetic lubricating oils comprise esters of dicarboxylic acids and those made from C5 to C12 monocarboxylic acids and polyols or polyol ethers. Other synthetic lubricating oils include liquid esters of phosphorus-containing acids, polymeric tetrahydrofurans, silicon-based oils such as poly-alkyl-, polyaryl-, poly- alkoxy-, or polyaryloxy-siloxane oils, and silicate oils.

Other synthetic oils include those produced by Fischer-Tropsch reactions, typically hydroisomerized Fischer-Tropsch hydrocarbons or waxes. In one embodiment oils may be prepared by a Fischer-Tropsch gas-to-liquid synthetic procedure as well as other gas-to-liquid oils.

Unrefined, refined, and rerefined oils, either natural or synthetic (as well as mixtures thereof) of the types disclosed hereinabove can used. Unrefined oils are those obtained directly from a natural or synthetic source without further purification treatment. Refined oils are similar to the unrefined oils except they have been further treated in one or more purification steps to improve one or more properties. Rerefined oils are obtained by processes similar to those used to obtain refined oils applied to refined oils which have been already used in service. Rerefined oils often are additionally processed to remove spent additives and oil breakdown products.

The amount of the oil of lubricating viscosity present in a lubricant is typically the balance remaining after subtracting from 100 wt % the sum of the amount of the compound of the invention and the other performance additives.

Lubricants prepared using the materials of the presently-disclosed technology will typically contain one or more additional additive of the types that are known to be used as lubricant additives. One such additive is a dispersant. Dispersants are well known in the field of lubricants and include primarily what is known as ashless-type dispersants and polymeric dispersants. Ashless type dispersants are characterized by a polar group attached to a relatively high molecular weight hydrocarbon chain. Typical ashless dispersants include nitrogen-containing dispersants such as N-substituted long chain alkenyl succinimides, also known as succinimide dispersants. Succinimide dispersants are more fully described in U.S. Pat. Nos. 4,234,435 and 3,172,892. Another class of ashless dispersant is high molecular weight esters, prepared by reaction of a hydrocarbyl acylating agent and a polyhydric aliphatic alcohol such as glycerol, pentaerythritol, or sorbitol. Such materials are described in more detail in U.S. Pat. No. 3,381,022. Another class of ashless dispersant is Mannich bases. These are materials which are formed by the condensation of a higher molecular weight, alkyl substituted phenol, an alkylene polyamine, and an aldehyde such as formaldehyde and are described in more detail in U.S. Pat. No. 3,634,515. Other dispersants include polymeric dispersant additives, which are generally hydrocarbon-based polymers which contain polar functionality to impart dispersancy characteristics to the polymer. Dispersants can also be post-treated by reaction with any of a variety of agents. Among these are urea, thiourea, dimercaptothiadiazoles, carbon disulfide, aldehydes, ketones, carboxylic acids, hydrocarbon-substituted succinic anhydrides, nitriles, epoxides, boron compounds, and phosphorus compounds. References detailing such treatment are listed in U.S. Pat. No. 4,654,403. The amount of dispersant in the present composition can typically be 1 to 10 weight percent, or 1.5 to 9.0 percent, or 2.0 to 8.0 percent, all expressed on an oil-free basis.

Another component is an antioxidant. Antioxidants encompass phenolic antioxidants, which may comprise a butyl substituted phenol containing 2 or 3 t-butyl groups. The para position may also be occupied by a hydrocarbyl group, an ester-containing group, or a group bridging two aromatic rings. Antioxidants also include aromatic amine, such as nonylated diphenylamines or (optionally alkylated) phenylnaphthylamine. Other antioxidants include sulfurized olefins, titanium compounds, and molybdenum compounds. U.S. Pat. No. 4,285,822, for instance, discloses lubricating oil compositions containing a molybdenum and sulfur containing composition. U.S. Patent Application Publication 2006-0217271 discloses a variety of titanium compounds, including titanium alkoxides and titanated dispersants, which materials may also impart improvements in deposit control and filterability. Other titanium compounds include titanium carboxylates such as neodecanoate. Typical amounts of antioxidants will, of course, depend on the specific antioxidant and its individual effectiveness, but illustrative total amounts can be 0.01 to 5 percent by weight or 0.15 to 4.5 percent or 0.2 to 4 percent. Additionally, more than one antioxidant may be present, and certain combinations of these can be synergistic in their combined overall effect.

Viscosity improvers (also sometimes referred to as viscosity index improvers or viscosity modifiers) may be included in the compositions of this invention. Viscosity improvers are usually polymers, including polyisobutenes, polymethacrylic acid esters, hydrogenated diene polymers, polyalkylstyrenes, esterified styrene-maleic anhydride copolymers, hydrogenated alkenylarene-conjugated diene copolymers and polyolefins. Multifunctional viscosity improvers, which also have dispersant and/or antioxidancy properties are known and may optionally be used.

Another additive is an antiwear agent. Examples of anti-wear agents include phosphorus-containing antiwear/extreme pressure agents such as metal thiophosphates, phosphoric acid esters and salts thereof, phosphorus-containing carboxylic acids, esters, ethers, and amides; and phosphites. In certain embodiments a phosphorus antiwear agent may be present in an amount to deliver 0.01 to 0.2 or 0.015 to 0.15 or 0.02 to 0.1 or 0.025 to 0.08 percent phosphorus. Often the antiwear agent is a zinc dialkyldithiophosphate (ZDP). For a typical ZDP, which may contain 11 percent P (calculated on an oil free basis), suitable amounts may include 0.09 to 0.82 percent. Non-phosphorus-containing anti-wear agents include borate esters (including borated epoxides), dithiocarbamate compounds, molybdenum-containing compounds, and sulfurized olefins.

Other materials that may be used as antiwear agents include tartrate esters, tartramides, and tartrimides. Examples include oleyl tartrimide (the imide formed from oleylamine and tartaric acid) and alkyl diesters (from, e.g., mixed C12-16 alcohols). Other related materials that may be useful include esters, amides, and imides of other hydroxy-carboxylic acids in general, including hydroxy-polycarboxylic acids, for instance, acids such as tartaric acid, citric acid, lactic acid, glycolic acid, hydroxypropionic acid, hydroxyglutaric acid, and mixtures thereof. These materials may also impart additional functionality to a lubricant beyond antiwear performance. These materials are described in greater detail in US Publication 2006-0079413 and PCT publication WO2010/077630. Such derivatives of (or compounds derived from) a hydroxy-carboxylic acid, if present, may typically be present in the lubricating composition in an amount of 0.1 weight % to 5 weight %, or 0.2 weight % to 3 weight %, or greater than 0.2 weight % to 3 weight %.

Other additives that may optionally be used in lubricating oils include pour point depressing agents, extreme pressure agents, anti-wear agents, color stabilizers and anti-foam agents.

Lubricants containing the materials of the disclosed technology may be used for the lubrication of a wide variety of mechanical devices, including internal combustion engines, both two-stroke cycle and four-stroke cycle, spark-ignited and compression-ignited, sump-lubricated or non-sump-lubricated. The engines may be run on a variety fuels including gasoline, diesel fuel, alcohols, bio-diesel fuel, and hydrogen, as well as mixtures of these (such as gasoline-alcohol mixtures, e.g., E-10, E-15, E-85). Examples include passenger-car gasoline engines, and passenger-car diesel engines, and heavy duty diesel engines.

The disclosed lubricants are suitable for use as lubricants for marine diesel engines, particularly as cylinder lubricants. In one embodiment, the present technology provides a method for lubricating an internal combustion engine, comprising supplying thereto a lubricant comprising the composition as described herein. The invention is suitable for 2-stroke or 4-stroke engines, in particular marine diesel engines, especially 2-stroke marine diesel engines.

The amount of each chemical component described is presented exclusive of any solvent or diluent oil, which may be customarily present in the commercial material, that is, on an active chemical basis, unless otherwise indicated. However, unless otherwise indicated, each chemical or composition referred to herein should be interpreted as being a commercial grade material which may contain the isomers, byproducts, derivatives, and other such materials which are normally understood to be present in the commercial grade.

As used herein, the term “hydrocarbyl substituent” or “hydrocarbyl group” is used in its ordinary sense, which is well-known to those skilled in the art. Specifically, it refers to a group having a carbon atom directly attached to the remainder of the molecule and having predominantly hydrocarbon character. Examples of hydrocarbyl groups include: hydrocarbon substituents, including aliphatic, alicyclic, and aromatic substituents; substituted hydrocarbon substituents, that is, substituents containing non-hydrocarbon groups which, in the context of this invention, do not alter the predominantly hydrocarbon nature of the substituent; and hetero substituents, that is, substituents which similarly have a predominantly hydrocarbon character but contain other than carbon in a ring or chain. A more detailed definition of the term “hydrocarbyl substituent” or “hydrocarbyl group” is found in paragraphs [0137] to [0141] of published application US 2010-0197536.

It is known that some of the materials described above may interact in the final formulation, so that the components of the final formulation may be different from those that are initially added. For instance, metal ions (of, e.g., a detergent) can migrate to other acidic or anionic sites of other molecules. The products formed thereby, including the products formed upon employing the composition of the present invention in its intended use, may not be susceptible of easy description. Nevertheless, all such modifications and reaction products are included within the scope of the present invention; the present invention encompasses the composition prepared by admixing the components described above.

### EXAMPLES

### Example 1

A 1000 Mₙ polyisobutylene-alkylated phenol (455 g, 0.416 moles) is heated to 100° C. while stirring in a vessel under a nitrogen blanket. Calcium hydroxide (19.4 g, 0.262 moles) and ethylene glycol (35.0 g) are added and the mixture is heated to 124° C. Sulfur (90.7 g, 2.834 moles) is added and the mixture is heated to 160° C. p-Cresol (45 g, 0.417 moles) is then added dropwise over 30 minutes while increasing the temperature to 195° C. The reaction mixture is held at this temperature for 4.5 hours. Diluent oil (107 g) is added and the mixture is then cooled to room temperature. The bridged phenolic product has a calculated average carbon number in the hydrocarbyl substituents of about 34.5.

Ethylene glycol (27.4 g) and n-decanol (64.3 g) are added to the reaction mixture with stirring, and the mixture is then heated to 168° C. under a nitrogen blanket. Calcium hydroxide (15.6 g, 0.166 moles) is then added and the mixture is heated to 220° C. while removing volatiles by distillation. At 220° C. a vacuum (13 kPa pressure, 27″ Hg vacuum) is applied to the reaction mixture for 1 hour. Additional diluent oil (113 g) is added and the mixture is cooled to 100° C. and then filtered to yield the final product, in diluent oil.

### Example 2

A 550 Mₙ polyisobutylene-alkylated phenol (428 g, 0.667 moles) is heated to 100° C. while stirring in a vessel under a nitrogen blanket. Calcium hydroxide (45.6 g, 0.628 moles) and ethylene glycol (84 g) are added and the mixture is heated to 124° C. Sulfur (217.6 g, 6.80 moles) is added and the mixture is heated to 160° C. p-Cresol (72 g, 0.667 moles) is then added dropwise over 30 minutes while increasing the temperature to 195° C. The reaction mixture is held at this temperature for 4.5 hours. Diluent oil (107 g) is added and the mixture is then cooled to room temperature. The bridged phenolic product has a calculated average carbon number in the hydrocarbyl substituents of about 20.

Ethylene glycol (27.4 g) and n-decanol (64.3 g) are added to the reaction mixture with stirring, and the mixture is then heated to 168° C. under a nitrogen blanket. Calcium hydroxide (9.47 g, 0.131 moles) is then added and the mixture is heated to 220° C. while removing volatiles by distillation. At 220° C. a vacuum (13 kPa pressure, 27″ Hg vacuum) is applied to the reaction mixture for 1 hour. Additional diluent oil (113 g) is added and the mixture is cooled to 100° C. and then filtered to yield the final product, in diluent oil.

### Examples 3 and 4

Each of the products of Examples 1 and 2 is added to a base oil, along with other, conventional, components to prepare a lubricating composition.

### Examples 5 and 6

Each of the products of Examples 1 and 2 is overbased by reaction with a molar excess of calcium hydroxide and the mixture blown with carbon dioxide to lead to solution of the components, thereby preparing an overbased detergent. Each of the overbased detergents is, separately, added to a base oil, along with other, conventional, components to prepare a lubricating composition.

### Example 7

Polyisobutylene phenol (445.0 g, prepared form polyisobutylene of about 1000 Mₙ, or about 70 carbon atoms), Ca(OH)₂ (19.4 g), ethylene glycol (35.0 g) and sulfur (90.7 g) are heated to 160° C. Para-cresol (45.0 g) is added while heating to 195° C. and maintained at temperature for 4.5 hours; then diluent oil (107 g) is added and the solution is cooled to 90° C. Ethylene glycol (21.6 g), decanol (50.7 g) and Ca(OH)₂ (12.3 g) are then added the mixture is subsequently stirred for 1 hour.

The mixture is then heated to 220° C. under full vacuum, 5 kPa (40 mm Hg), and maintained at this temperature for 1 hour. Diluent oil (89.2 g) is added to the mixture, which is then cooled to room temperature. The final product is obtained through filtration to remove residual solids, yielding a brown liquid (549.7 g).

### Example 8

Polyisobutylene phenol as in Example 7 (192.6 g), Ca(OH)₂ (8.6 g), ethylene glycol (15.5 g) and sulfur (40.2 g) are heated to 160° C. Para-cresol (28.8 g) is added while heating to 195° C. and held for 4.5 hrs; then diluent oil (47.4 g) is added and the solution is cooled to 90° C. Ethylene glycol (12.1 g), decanol (28.4 g) and Ca(OH)₂ (7.0 g) are then added and the mixture is subsequently stirred for 1 hour. The mixture is then heated to 220° C. under full vacuum 5 kPa (40 mm Hg) and held at this temperature for 1 hour. Diluent oil (50.0 g) is added to the mixture, which is then cooled to room temperature. The final product is obtained through filtration to remove residual solids to yield a brown liquid (243.0 g).

Lubricants containing the phenolic compounds of Examples 7 and 8, and, for comparison, containing a comparable amount of a calcium salt of sulfur-bridged p-dodecylphenol are subject to the Komatsu Hot Tube (KHT) test. This is an industry test used to evaluate performance of engine oils based on their deposit-forming tendencies by circulating a sample of the engine oil at 0.31 mL per hour and air at 10 mL per minute through a glass tube for 16 hours at a specified temperature, in this instance at 320° C. After the test, the tubes are visually rated, with a higher number being a better rating: 10 representing a clean tube and 0 (zero) representing a tube with heavy deposits.

The lubricant base formulation contains about 8.9% overbased calcium sulfonate detergent (oil free amount), about 0.7% (oil free) of a succinimide dispersant and 0.03% of a commercial antifoam agent, and 5.0% (oil-containing) of the phenate detergent as shown, in mineral oil:

| | | | Commercial |
|---|---|---|---|
| | | | sulfurized Ca phenate |
| | | | detergent, PDDP- |
| 5.0 weight | Ex. 7 (includes | Ex. 8 (includes | based, 27% |
| % of: | about 16% oil) | about 20% oil) | diluent oil |
| 320° C. KHT | 7.5 | 7.5 | 8.5 |
| Rating: | | | |

Each of the lubricants of Examples 7 and 8 gives comparable results in terms of tube cleanliness rating with that of the sulfur-bridged p-dodecylphenol salt, even though they contain no p-dodecylphenol component.

For comparison, in a similar lubricant base formulation, another similar commercial sulfurized Ca phenate detergent, PDDP-based, gives a 320° C. KHT rating of 7.5. That reference example used a treat rate of 17.5% phenate (including 39% oil). This indicates that the phenates of the disclosed technology, even at a lower phenate substrate level, can give equivalent performance to that of a conventional phenate.

Each of the documents referred to above is incorporated herein by reference, including any prior applications, whether or not specifically listed above, from which priority is claimed. The mention of any document is not an admission that such document qualifies as prior art or constitutes the general knowledge of the skilled person in any jurisdiction. Except in the Examples, or where otherwise explicitly indicated, all numerical quantities in this description specifying amounts of materials, reaction conditions, molecular weights, number of carbon atoms, and the like, are to be understood as modified by the word “about.” It is to be understood that the upper and lower amount, range, and ratio limits set forth herein may be independently combined. Similarly, the ranges and amounts for each element of the invention can be used together with ranges or amounts for any of the other elements. As used herein, the expression “consisting essentially of” permits the inclusion of substances that do not materially affect the basic and novel characteristics of the composition under consideration.

1. A bridged dimeric or oligomeric phenolic compound comprising:
at least one monomer unit (a) of phenol or an alkyl-substituted phenol wherein the alkyl group contains 1 to 8 carbon atoms, or mixtures thereof;
at least one monomer unit (b) of an aliphatic hydrocarbyl-substituted phenol wherein the aliphatic hydrocarbyl group contains at least about 25 carbon atoms, or mixtures thereof; and
at least one sulfur-containing bridging group;
or a salt of said oligomeric material;
wherein the average number of carbon atoms in said alkyl groups and said aliphatic hydrocarbyl groups is about 10 to about 100.

2. The bridged dimeric or oligomeric phenolic compound of claim 1 being substantially free of monomer units of C₁₂-alkyl phenol.

3. The bridged dimeric or oligomeric phenolic compound of claim 1 containing one or more bridging groups of one or more sulfur atoms, or mixtures thereof.

4. The bridged dimeric or oligomeric phenolic compound of claim 1 wherein the oligomeric material is represented by the structure 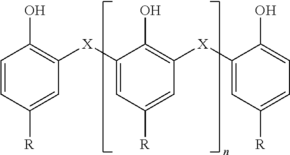
or isomers thereof, wherein:
each R is independently hydrogen or an aliphatic hydrocarbyl group, provided that at least one R represents an aliphatic hydrocarbyl group containing at least about 25 carbon atoms and at least one R represents hydrogen or a hydrocarbyl group of 1 to about 4 carbon atoms;
each X is independently a bridge of one or more sulfur atoms represented by Sₓ where x is 1 to 4; and
n is 0 to about 8.

5. The bridged dimeric or oligomeric phenolic compound of claim 1 wherein the oligomeric material is represented by the structure 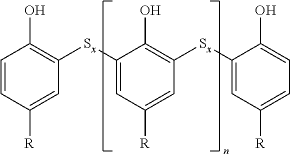
wherein:
each R is independently hydrogen or an aliphatic hydrocarbyl group,
provided that at least one R represents an aliphatic hydrocarbyl group containing at least about 25 carbon atoms and at least one R represents hydrogen or a hydrocarbyl group of 1 to about 4 carbon atoms;
Sₓ represents a bridge of one or more sulfur atoms, where each x is independently 1 or 2; and
n is 1 to 6.

6. The bridged dimeric or oligomeric phenolic compound of claim 4 wherein n is 1 to 6 and wherein one or two R groups are aliphatic hydrocarbyl groups containing about 30 to about 200 carbon atoms and the remaining R groups are methyl.

7. The bridged dimeric or oligomeric phenolic compound of claim 1 wherein the aliphatic hydrocarbyl group containing at least about 25 carbon atoms is an oligomer or polymer of propylene or of isobutylene.

8. The bridged dimeric or oligomeric phenolic compound of claim 1 being in the form of a metal salt.

9. The bridged dimeric or oligomeric phenolic compound of claim 8 wherein the metal salt is a calcium salt, a magnesium salt, or a sodium salt.

10. The bridged dimeric or oligomeric phenolic compound of claim 1 being in the form of an overbased metal salt.

11. The bridged dimeric or oligomeric phenolic compound of claim 1, being substantially free of monomer units of unsubstituted phenol.

12. A product prepared by reacting at least one monomer (a) of phenol or an alkyl-substituted phenol wherein the alkyl group contains 1 to 8 carbon atoms, or mixtures thereof; and
at least one monomer (b) of an aliphatic hydrocarbyl-substituted phenol wherein the aliphatic hydrocarbyl group contains at least about 25 carbon atoms, or mixtures thereof;
with a bridging agent comprising sulfur or an aldehyde or ketone,
wherein the amounts of (a) and (b) are such that the average number of carbon atoms in said alkyl groups and said aliphatic hydrocarbyl groups is about 10 to about 100.

13. The product of claim 12 wherein the phenol or alkyl-substituted phenol of component (a) comprises an alkyl-substituted phenol wherein the alkyl group contains 1 to 4 carbon atoms.

14. (canceled)

15. A lubricating composition comprising an oil of lubricating viscosity and a bridged dimeric or oligomeric phenolic compound of claim 1.

16. The lubricating composition of claim 15 further comprising at least one of detergents other than bridged dimeric or oligomeric phenolic compounds, dispersants, antiwear agents, antioxidants, zinc dialkyldithiophosphates, viscosity modifiers, pour point depressing agents, and anti-foam agents.

17. The lubricating composition of claim 15 further comprising an overbased calcium sulfonate detergent having a TBN (oil-free basis) of at least about 300.

18. A method of lubricating a mechanical device, comprising supplying thereto the lubricant composition of claim 15.

19. The method of claim 18 wherein the mechanical device comprises an internal combustion engine.
